SEQUENCE LISTING (37 sequences)

SEQ ID NO: 1 - DNA, 15 bp, Artificial
  Xcm I recognition sequence
ccannnnnnnnntgg

SEQ ID NO: 2 - DNA, 15 bp, Artificial
  barcode adaptor first strand
ccannnntnnnntgg

SEQ ID NO: 3 - DNA, 15 bp, Artificial
  barcode adaptor opposite strand
ggtnnnnannnnacc

SEQ ID NO: 4 - DNA, 15 bp, Artificial
  preferred barcode adaptor first strand
ccannnntnnnntgg

SEQ ID NO: 5 - DNA, 15 bp, Artificial
  preferred barcode adaptor opposite strand
ggtnnnnannnnacc

SEQ ID NO: 6 - DNA, 11 bp, Artificial
  Eam1105I recognition sequence
gacnnnnngtc

SEQ ID NO: 7 - DNA, 11 bp, Artificial
  barcode adaptor, first strand
gacnntnngtc

SEQ ID NO: 8 - DNA, 11 bp, Artificial
  barcode adaptor, opposite strand
gacnnanngtc

SEQ ID NO: 9 - DNA, 11 bp, Artificial
  preferred barcode adaptor, first strand
gacnntnngtc

SEQ ID NO: 10 - DNA, 11 bp, Artificial
  preferred barcode adaptor, opposite strand
gacnnanngtc

SEQ ID NO: 11 - DNA, 33 bp, Artificial
  PE Adapter1 first strand
acactctttccctacacgacgctcttccgatct

SEQ ID NO: 12 - DNA, 32 bp, Artificial
  PE Adapter1 opposite strand
gatcggaagagcgtcgtgtagggaaagagtgt

SEQ ID NO: 13 - DNA, 32 bp, Artificial
  PE Adapter2 first strand
gatcggaagagcggttcagcaggaatgccgag

SEQ ID NO: 14 - DNA, 33 bp, Artificial
  PE Adapter2 opposite strand
ctcggcattcctgctgaaccgctcttccgatct

SEQ ID NO: 15 - DNA, 58 bp, Artificial
  PE PCR Primer1
aatgatacggcgaccaccgagatctacactctttccctacacgacgctcttccgatct

SEQ ID NO: 16 - DNA, 61 bp, Artificial
  PE PCR Primer2
caagcagaagacggcatacgagatcggtctcggcattcctgctgaaccgctcttccgatct

SEQ ID NO: 17 - DNA, 120 bp, Artificial
  Result library first strand
aatgatacggcgaccaccgagatctacactctttccctacacgacgctcttccgatctnagatcggaagagcggttcagcaggaatgccgagaccgatctcgtatgccgtcttctgcttg

SEQ ID NO: 18 - DNA, 120 bp, Artificial
  Result library opposite strand
caagcagaagacggcatacgagatcggtctcggcattcctgctgaaccgctcttccgatctnagatcggaagagcgtcgtgtagggaaagagtgtagatctcggtggtcgccgtatcatt

SEQ ID NO: 19 - DNA, 33 bp, Artificial; identical to SEQ ID NO: 11
  PE DNA Sequencing Primer1

SEQ ID NO: 20 - DNA, 37 bp, Artificial
  PE DNA Sequencing Primer2
cggtctcggcattcctgctgaaccgctcttccgatct

SEQ ID NO: 21 - DNA, 33 bp, Artificial; identical to SEQ ID NO: 11
  Adapter first strand

SEQ ID NO: 22 - DNA, 32 bp, Artificial; identical to SEQ ID NO: 12
  Adapter opposite strand

SEQ ID NO: 23 - DNA, 33 bp, Artificial
  Adapter first strand
gatcggaagagctcgtatgccgtcttctgcttg

SEQ ID NO: 24 - DNA, 34 bp, Artificial
  Adapter opposite strand
caagcagaagacggcatacgagctcttccgatct

SEQ ID NO: 25 - DNA, 58 bp, Artificial; identical to SEQ ID NO: 15
  PCR Primer

SEQ ID NO: 26 - DNA, 34 bp, Artificial; identical to SEQ ID NO: 24
  PCR Primer

SEQ ID NO: 27 - DNA, 93 bp, Artificial
  Result library first strand
aatgatacggcgaccaccgagatctacactctttccctacacgacgctcttccgatctnagatcggaagagctcgtatgccgtcttctgcttg

SEQ ID NO: 28 - DNA, 93 bp, Artificial
  Result library opposite strand
caagcagaagacggcatacgagctcttccgatctnagatcggaagagcgtcgtgtagggaaagagtgtagatctcggtggtcgccgtatcatt

SEQ ID NO: 29 - DNA, 33 bp, Artificial; identical to SEQ ID NO: 11
  Genomic DNA Sequencing Primer

SEQ ID NO: 30 - DNA, 41 bp, Artificial
  P1 Adapter first strand
ccactacgcctccgctttcctctctatgggcagtcggtgat

SEQ ID NO: 31 - DNA, 43 bp, Artificial
  P1 Adapter opposite strand
atcaccgactgcccatagagaggaaagcggaggcgtagtggtt

SEQ ID NO: 32 - DNA, 25 bp, Artificial
  P2 Adapter first strand
agagaatgaggaacccggggcagtt

SEQ ID NO: 33 - DNA, 23 bp, Artificial
  P2 Adapter opposite strand
ctgccccgggttcctcattctct

SEQ ID NO: 34 - DNA, 28 bp, Artificial
  Library PCR Primer1
ccactacgcctccgctttcctctctatg

SEQ ID NO: 35 - DNA, 23 bp, Artificial
  Library PCR Primer2
ctgccccgggttcctcattctct

SEQ ID NO: 36 - DNA, 65 bp, Artificial
  Resulting Frag Library first strand
ccactacgcctccgctttcctctctatgggcagtcggtgatnagagaatgaggaacccggggcag

SEQ ID NO: 37 - DNA, 65 bp, Artificial
  Resulting Frag Library opposite strand
ctgccccgggttcctcattctctnatcaccgactgcccatagagaggaaagcggaggcgtagtgg

(19) United States
(12) Patent Application Publication
(10) Pub. No.: US 2012/0071327 A1
(43) Pub. Date: Mar. 22, 2012

(54) INDEXING OF NUCLEIC ACID POPULATIONS

(75) Inventors: Peer F. Staehler, Mannheim (DE); Cord F. Staehler, Weinheim (DE); Markus Beier, Weinheim (DE); Mark S. Chee, Del Mar, CA (US); Nadine Schracke, Hirschberg (DE)

(73) Assignee: FEBIT HOLDING GMBH, Heidelberg (DE)

(21) Appl. No.: 13/139,327

(22) PCT Filed: Dec. 11, 2009
(86) PCT No.: PCT/EP2009/066949

§ 371 Date: Oct. 31, 2011

Related U.S. Application Data
(60) Provisional application No. 61/121,615, filed on Dec. 11, 2008.

(30) Foreign Application Priority Data
Dec. 11, 2008 (DE) 10 2008 061 774.1

(51) Int. Cl. C40B 20/00; C07H 1/06; C40B 60/10; C40B 30/04; C12M 1/40; C07H 21/00; G01N 33/53

(52) U.S. Cl. 506/2; 536/25.4; 436/501; 506/9; 422/69; 435/287.2; 506/38

(57) ABSTRACT

The invention relates to a method for acquisition of genetic information, in particular for personalized medicine.

The invention relates to a method for acquisition of genetic information, in particular for personalized medicine.

Acquisition of genetic information is a central process in molecular diagnostics. From economic aspects, this acquisition should be as inexpensive as possible. From diagnostic, medical, regulatory and ethical aspects, this acquisition should be as accurate as possible and rule out falsely positive measurements.

The use of genetic information, that is to say information in the genetic material, is undisputedly already of great value nowadays. It will also be attributed even more value in the future, since further knowledge is generally expected for the use of genetic information in medical treatment. Apart from the human genetic material, including the mitochondria, this interest also applies in particular to the genetic material of pathogens and organisms which cause diseases.

Alongside the medical field of use, fields of use which benefit from improved acquisition of genetic information also additionally exist in other areas of biotechnology.

In addition to traditional Sanger sequencing, which is still the gold standard of genome analysis, sequencing technologies have become available which have a very much higher performance compared with Sanger and redefine the term ultra-high throughput DNA sequencing. Several sequencing platforms of this next generation, which are also called “next generation sequencing” or NGS, are known to the person skilled in the art.

The new sequencing technologies allow acquisition of genetic information by an open system of DNA sequencing instead of resorting to closed analysis systems, such as, for example, microarrays. It is thus possible, for example, to detect very rare somatic changes in the genome of single cells in complex cell populations by sequencing, which contributes inter alia to elucidation of tumor formation. The lower costs per DNA base compared with Sanger sequencing now allow sequencing projects which were hitherto economically difficult, such as e.g. characterization of industrial production strains in biotechnology, to be undertaken.

Technologically, a common feature of the new methods is that instead of cloning into bacterial or viral systems for multiplication of single DNA sequences, a direct clonal amplification of DNA single molecules takes place, these having to be suitably prepared in the overall process. Compact instruments with automated processes replace expensive laboratory processes, and functionalized surfaces and in vitro methods replace biological systems.

The SOLiD platform of Applied Biosystems/Life Technologies is based on sequencing by oligonucleotide ligation and detection. It is a system of the next generation for DNA analysis with a very high throughput. In contrast to polymerase-based sequencing methods, the SOLiD system uses a technology called “stepwise ligation”. Single molecules bonded to particles are a central element of the system called Roche-454, which replace bacterial clones. These single molecules are amplified clonally in a particular formate of the PCR—emulsion PCR—and are subsequently distributed over picotiter plates with several hundred thousand wells and then sequenced by means of pyrosequencing, which is known and published in the field.

A further method known to the person skilled in the art uses so-called “clonal single molecule arrays” in a flow cell, onto which up to 40 million DNA single molecules can be covalently bonded. This technology is marketed by Illumina.

Amplification of the single strands takes place here via so-called “bridge amplification”, in which spatially separate, covalently bonded copy clusters, also called “polonies”, are formed on a surface. The sequencing itself is based on the “sequencing-by-synthesis method” with fluorescence-labeled nucleotides. The nucleotides incorporated have reversibly blocked 3′ groups on the bases, which are each removed at precisely coordinated times in each process cycle, so that incorporation and reading is performed nucleotide for nucleotide. Resolution of homopolymers is therefore also good. As a characteristic number, 40 million reading results (reads) with lengths of up to 35 nucleotides (so-called micro-reads) can be achieved and then in their entirety deliver up to 1,000 Mb (1 Gb) of sequence information in only a single sequencing run in one apparatus.

All the sequencing methods of the next generation known to the person skilled in the art and those described here have the common feature of the difficulty of sequencing a sample of more than 10 megabases of DNA in total size in one sequencing run. Access to a sufficiently small part of a complex genome of more than 10 megabases of DNA in total size also cannot be achieved by the methods described alone.

Methods for enrichment of desired target molecules in a nucleic acid population based on a solid matrix (e.g. microarrays, beads) or a liquid matrix (nucleic acid libraries in solution) exist. Enrichment methods by means of a large number of PCRs performed in parallel are furthermore also known. Such methods are described e.g. in U.S. Pat. No. 6,013,440, U.S. Pat. No. 6,632,611, U.S. Pat. No. 7,214,490, DE 101 49 947 and U.S. Pat. No. 7,320,862, WO 2007/057652, WO 2008/115185, US 2008/194413, P. Parameswaran, Nucleic Acid Research, 2007, 35(19), e130, M, Meyer, Nucleic Acid Research, 2007, 35(15), e97, E. Hodges, Nature Genetics, 2007, 39(12):1522-7, T. Albert, Nature Methods, 2007, 4(11):903-5, or D. W. Craig, Nat Methods, 2008 October; 5(10):887-93.

Selective extraction of parts of a genome with the aid of specific sequences present therein is also described in WO 2003/031965 and DE 10 2007 056 398.3, to the disclosure of which reference is made herewith.

A further embodiment of an extraction method is known to the person skilled in the art under the term “Hybselect”. Other embodiments are called “sequence capture” and “genome partitioning”, “enrichment”, “selection for regions of interest (ROI)”.

The Hybselect method preferentially uses capture probes on a solid phase. In a specific embodiment, a DNA microarray in a microfluidic biochip is used for sequence-dependent bonding and extraction of DNA. The biochip is thus employed preparatively. One field of use is the use of Hybselect for enrichment of DNA for massively parallel sequencing apparatuses.

Hybselect achieves as the central object the necessary rescaling of complex genomes, so that these can be processed and analyzed as a sample by an NGS apparatus. In the case of the Genome Analyzer (GA) 2 from Illumina, this means instantaneously a rescaled “complexity” of less than 10 megabases in the individual sample.

By rescaling of complex genomes, Hybselect makes targeted analysis of any desired selection of genomic sequences (random access) for resequencing possible. The NGS system can finally process genomic samples in a targeted manner. The throughput of the NGS system is utilized to the optimum.

Without Hybselect, on the other hand, only the entire genome can be resequenced with the NGS of status 2008. The company Illumina has done precisely this for a Yoruba man from the 1,000 genome study with the following characteristic values: cost 100,000 USD, duration 8 weeks, team of 150 members (published in Nature in November 2008), employing min. 5 Genome Analyzer apparatuses.

For use for example in clinical studies and translational genomics in oncology, that means access to several megabases of sequence information per patient for hundreds of patients on one NGS system coupled with a Hybselect system. This sequence information can be, inter alia, oncogenes, known mutation hotspots or regulatory sequences.

Only by combination of the two technologies (Hybselect and NGS) does it become possible to obtain defined sequence information for statistically relevant numbers of patients.

The invention is based on the problem of making the acquisition of genetic information less expensive, more simple, more reliable and more efficient compared with the prior art.

For this, the process of acquisition of genetic information is broken down into two steps. In the first step an enrichment is carried out, in which target regions in the genome or in the sample material are enriched according to sequence. In the second step sequencing of the enriched sample is performed.

The invention provides the analysis of nucleic acid populations. The invention thus relates to methods for isolation of target nucleic acid molecules, comprising the steps:
(a) providing one or more nucleic acid molecule populations to be analyzed,
(b) introducing markings into the nucleic acid populations to be analyzed,
(c) bringing the one or more populations of nucleic acid molecules into contact with capture molecules under conditions under which target nucleic acid molecules from the population or populations to be analyzed can bind specifically to the capture molecules,
(d) separating off material not bound to capture molecules and
(e) isolating and optionally characterizing the target nucleic acid molecules isolated, comprising determination of the markings.

In contrast to conventional methods, the nucleic acids of a nucleic acid population to be analyzed (the sample) are provided, as part of the preparation (sample preparation), with specific markings (or labels) which are suitable for a characterization which is independent of the sequence of the sample. By these markings, each sample is given a molecular “bar code”. This method makes common process steps with several samples in a mixture possible, and therefore contributes towards increasing the efficiency, and moreover the method reduces costs for equipment and for reagents. Furthermore, the use of such markings makes it possible to monitor the method procedure. They allow assignment to important process data/parameters, inter alia to the laboratory performing the method, the batch of the reagents, the time of the sequencing run, assignment to an experimenter or operator and the use of further technical equipment for more than one sample. Accordingly, a barcode is assigned to the most important parameters (e.g. the laboratory, the person conducting the experiment, the operator, the sequencing device, the reagent batch, the sequencing run, the sequencing carrier, the sequencing space/channel/subspace, the sequencing laboratory, etc.) when performing the method. This marking may later be used for the correlation of the parameters with the sequencing result.

Since marking of the nucleic acid population to be analyzed makes acquisition and differentiation of the sample and entrained material possible, a novel, improved state of data quality and robustness can be achieved. This acquisition of sample and entrained material and the assignment of samples to space and time coordinates, such as a laboratory or a time corridor, based on this is novel and of great advantage compared with the prior art for use of sequencing as a diagnostic method.

The nucleic acid populations to be analyzed can originate from a eukaryotic species, e.g. a mammalian species, such as, for example, humans, a prokaryotic species, such as, for example, a bacterium, or a viral species or a mixture of such nucleic acid populations. Preferably, mixtures of at least two nucleic acid populations are analyzed.

The mixtures of nucleic acid populations to be analyzed comprise at least two different populations which differ with respect to their source (e.g. species, organism, individual) and/or with respect to their complexity or fragment size and/or with respect to other parameters (e.g. the laboratory, the person conducting the experiment, the operator, the sequencing device, the reagent batch, the sequencing run, the sequencing carrier, the sequencing space/channel/subspace, the sequencing laboratory, etc.). The populations can originate from eukaryotic species, e.g. mammalian species, such as, for example, humans, or prokaryotic species, such as, for example, a bacterium, or viral species, or mixtures of eukaryotic or prokaryotic or viral species. The various nucleic acid populations can be those of the same species, but also those from different species. The populations can also originate from various organisms of one species, e.g. various human individuals. According to the invention, more than two different populations of nucleic acid molecules can also be analyzed, e.g. 3, 4, 5, 6 or even more populations.

In some embodiments, a nucleic acid population comprises at least 10²¹ different sequences, in other embodiments at least 10¹⁸ different sequences and in some embodiments up to 10¹⁵ different sequences, in other embodiments up to 10¹² different sequences, in other embodiments up to 10⁹ different sequences, in other embodiments up to 10⁶ different sequences, in other embodiments up to 10³ different sequences. The average length of individual sequences of the population can typically be about 20-20,000 nucleotides, e.g. about 100-10,000 nucleotides, for example about 100-600 or about 100-400 nucleotides. In certain embodiments populations of large fragments of typically about 5,000-20,000, e.g. about 8,000-15,000 nucleotides can typically be employed. The nucleic acids of a population can comprise double-stranded or single-stranded DNA, RNA or mixtures thereof.

The nucleic acid populations are preferably non-fragmented or obtainable by fragmentation of chromosomal or extrachromosomal DNA from one or more organisms, e.g. by enzymatic fragmentation, chemical fragmentation, mechanical fragmentation, such as, for example, by ultrasound treatment, or other methods.

A further improvement in the method is possible by consecutive isolation of target molecules in several successive cycles. In this case, the sample to be analyzed is brought into contact several times in succession with capture molecules, each of which can be identical or different.

The method according to the invention relates to the isolation of target molecules from two or more nucleic acid populations. The target molecules are conventionally subpopulations of the nucleic acid populations to be analyzed. For example, 10⁵ to 50×10⁶ and preferably 2×10⁵ to 10⁶ different target molecules can be isolated by the method according to the invention. The number of target molecules to be isolated correlates with the length of the regions of the nucleic acid sequences covered by capture probes. Typical ranges of the nucleic acid sequences which are isolated are 10 kb to 100 Mb, preferably 250 kb to 10 Mb, very preferably 500 kb to 4 Mb.

Capture molecules are used for isolation of the target molecules. These are nucleic acid molecules which bind specifically to the target molecules to be isolated, in particular by hybridization in the form of a nucleic acid double strand. The capture molecules are conventionally hybridization probes which are complementary, or at least complementary in partial regions, to the target molecules to be isolated. According to the invention, so-called wobble bases (inter alia degenerated bases, abasic sites, universal bases) which are complementary to more than one nucleic acid fragment can also be introduced into the capture probes. The hybridization probes can likewise be nucleic acids, in particular DNA or RNA molecules, but also nucleic acid analogues, such as peptide nucleic acids (PNA), locked nucleic acids (LNA) etc. The hybridization probes preferably have a length corresponding to 10-100 nucleotides and do not have to consist uninterruptedly of units with bases, i.e. they can also contain, for example, abasic units, linkers, spacers etc.

In the method according to the invention, the capture molecules can be immobilized on an array on particles (beads) or can be present in the free form, i.e. in solution.

The nucleic acid capture molecules used in the method according to the invention are preferably a population of at least 10, in some embodiments of at least 1,000, in other embodiments of at least 100,000, in other embodiments of at least 10,000,000 different nucleic acid molecules.

Sequences of nucleic acid capture molecules can be derived from databases or Internet databases or genome project databases which contain the nucleic acid sequences of organisms which have already been thoroughly sequenced. Alternatively, the sequences of nucleic acid capture molecules can also be chosen from as yet still unknown sequences, e.g. sequences which are not yet known in the nucleic acid populations to be analyzed.

The capture molecules used in the method according to the invention can be chosen such that they contain sequences of one or more of the nucleic acid molecule populations to be analyzed. In certain embodiments, capture molecules which recognize target molecules from not all of the nucleic acid populations to be analyzed can be chosen, for example capture molecules which recognize only target molecules from one of the nucleic acid populations to be analyzed.

According to the present invention, the nucleic acid molecule populations to be analyzed carry markings (or labels). Markings can be detectable groups, for example dyestuffs, fluorescence groups or partners of binding pairs which have bioaffinity, for example haptens, which bind specifically to antibodies, biotin, which binds specifically to avidin or streptavidin, or carbohydrates, which bind specifically to lectins.

A marking which represents a bar code which can be read by the sequencing technology is particularly preferred. According to the invention, this type of marking can be one or more terminal adaptor nucleic acid sequences. One part of the adaptor nucleic acids can, for example, make an amplification possible in subsequent steps, and another part of the adaptor nucleic acids can be the bar code which can be read later during the sequence analysis.

In a special embodiment of the present invention a marker/barcode is assigned to a given nucleic acid population according to the following steps:
a) fragmenting a given DNA/RNA-population
b) repairing the ends and adding overhangs, e.g. 3′A-overhangs
c) ligating barcode adaptors to the overhangs and
d) digesting with a restriction enzyme to produce overhangs, e.g. 3′-A-overhangs
e) ligating sequencing adaptors.

The standard procedure for sample preparation for a fragment library to be sequenced on an Illumina next generation sequencing system follows sequentially steps a), b) and e). The outlined procedure of the present invention following sequentially steps a), b), c), d) and e) has the advantage over the described prior art that specific restriction enzymes may be implemented in step d) in order to produce an overhang, e.g. an 3′-A-overhang that is already present in step b). Therefore, the incorporation of marker/barcode in step c) in combination with restriction digest in step d) is also orthogonal to the standard sample preparation procedure. In a preferred embodiment, barcode adaptors are nucleic acid double strands having a length from 10-100 nucleotides, particularly from 10-50 nucleotides, more particularly from 12-45 nucleotides. Advantageously, they have an overhang on at least one end, particularly a 3′-overhang. The overhang has a length of from 1-5 nucleotides, preferably 1 nucleotide, e.g. an A-overhang. Preferably, the barcode adaptors comprise a restriction enzyme recognition site and at least 1, preferably at least 2, e.g. 2, 3, 4 or 5, barcode positions, i.e. positions at which a nucleotide sequence characteristic for a predetermined parameter is present.

Example 2 and 3 describe the incorporation of especially preferred marker/barcodes by use of the present invention.

In a parallel analysis of several of the nucleic acid populations to be analyzed, the individual nucleic acid populations preferably carry different markings. In the context of isolation and optionally characterization of the nucleic acid target molecules, these can thus be assigned to a particular nucleic acid population, corresponding e.g. to an individual, a laboratory or a sequencing apparatus. The method according to the invention can contain a single isolation step or several cycles of consecutive isolation and optionally characterization of target molecules. The characterization of the target molecules in this context preferably comprises partial or complete determination of the sequences of the nucleic acid target molecules isolated.

In the context of an isolation procedure comprising several cycles, an amplification and/or a fragmentation of the target molecule population can be carried out between individual cycles.

In a further embodiment of the present invention, when the nucleic acid populations are brought into contact with the capture molecules, a DNA binding protein, in particular a DNA binding protein with an ATPase activity dependent on single-stranded DNA, such as, for example, RecA and optionally ATP, is added.

In certain embodiments of the method, an enrichment of target molecules using a capture probe matrix, e.g. a matrix of capture molecules bound to a solid phase, such as, for example, a biochip, is carried out as part of the preparation of the sample. As a particular advantage of the method according to the invention, the capture probe matrix can be used several times with or without purification or regeneration, since a differentiation between consecutive enrichments can be made on the basis of the different markings/bar codes used.

For this, the process of acquisition of the genetic information is broken down into two steps. In the first step an enrichment with marked sample material (sample 1) is carried out, in which, according to sequence, target regions in the sample material are bound to a microarray of nucleic acids using a capture probe matrix, e.g. a biochip, and are then eluted. The sequence analysis then takes place in a second step, preferably on a high throughput sequencing apparatus. After the sequence analysis, the data are assigned on the basis of the marker/bar code used.

If the identical target regions in the DNA are to subsequently be enriched for further sample material (sample 2), the capture probe matrix used beforehand can be employed again. In order to carry out a second consecutive enrichment on the same matrix, according to the invention either the matrix can first be purified, in order to remove traces of sample 1 still present, or, likewise according to the invention, purification can be omitted. Sample 2 is provided with a different marker (bar code) compared with sample 1. During the following sequence analysis of the sample 2 enriched in the target regions, with the aid of the bar codes a distinction can be very easily made between data originating from sample 2 and data originating from residues of sample 1.

It is known to the person skilled in the art that the process procedure described above is not limited only to enrichment on a microstructured biochip, but the capture probes used for enrichment of a target region can be provided generally on a solid phase of the most diverse materials (inter alia particles, microtiter plates, membranes, dip-stick assays etc.) or in the liquid phase.

The present invention links systems for high throughput sequencing, e.g. next generation sequencing: Roche-454, ABI-Solid, Illumina-Genome Analyzer, methods for sequence enrichment (e.g. WO 2003/031965, DE 10 2007 056 398.3) and methods for marking nucleic acid samples which make multiplexing possible, to give an efficient method which for the first time allows medically relevant parameters to be determined in a focused manner with a high throughput and acceptable costs.

By combination of this method with a multiple use, made possible via the marking, of the enrichment matrix (i.e. the capture molecules), the costs can moreover be lowered still further, or alternatively the range of determination of the focused medical parameters to be increased.

It was hitherto only possible to completely sequence the genomes of a few individuals. Even for this, an enormous amount of time and immense costs were required.

With the present invention it becomes possible for the first time to analyze statistically relevant cohorts of individuals with respect to defined medical parameters with acceptable costs and in a very short time. This is really considerable progress in the direction of personalized medicine.

The possibilities of quality control described are a further important aspect of the present invention. Since next generation sequencing involves very meticulous methods and instruments, it is particularly important here to establish corresponding quality standards. The present invention makes it possible to monitor the complete flow of the process from preparation of the sample to be analyzed to the analytical data via the coding/marking. As described, not only can the sequence data obtained be traced back in this way to the sequencing machines, to the laboratory and to the individual, further parameters can be acquired via the coding/marking, such as e.g. batches of chemicals, batches of the sample preparation kits, operators during the sample preparation, operators during the sequencing, batches of the enrichment matrices (biochips) etc. The person skilled in the art is able to name further process parameters which are important for the particular individual determination of individual medical parameters and to insert these into the coding/marking. Such a method of approach is of central importance precisely in view of certification before the appropriate health authorities (inter alia the FDA).

Preferred embodiments of the invention are explained in detail in the following.

In one embodiment, the nucleic acid sample(s) to be analyzed is/are indexed by a marking. The marking serves for later assignment of the sequence data to the corresponding individual or the corresponding experiment. The markings are preferably bar codes which can be read with the aid of a sequence analysis.

However, marking methods which allow decoding without sequence analysis are also possible, e.g. via dyestuffs or fluorescence codes.

Such a method for acquisition of information in the DNA or RNA of an individual comprises the steps:
selection of target regions in a DNA or RNA population,
preparation of the nucleic acid population of the individual for a sequence enrichment with addition of a marking which later allows assignment to the individual,
sequence-specific enrichment of target regions from the nucleic acid population, e.g. in/on a preparative biochip (or on beads or in the liquid phase), with corresponding capture molecules,
sequencing of the enriched target regions, comprising acquisition of the marking.

In a further embodiment, the genetic information of two or more individuals, e.g. human individuals, is acquired. The marking here allows assignment of the sequence data to the corresponding individuals. According to the invention, the enrichment of two or more individuals can therefore be carried out in parallel. That is to say the enrichment is carried out in a mixture of samples of the two or more individuals.

Such a method for acquisition of information in the DNA or RNA of at least two individuals comprises the steps:
selection of target regions in a DNA or RNA population,
preparation of nucleic acid populations of the individuals for a sequence enrichment with addition of a marking which later allows assignment to the individual,
sequence-specific enrichment of target regions from the nucleic acid populations of the two or more individuals, e.g. in/on a preparative biochip, such as, for example, a microfluid biochip (or on beads or in the liquid phase), with corresponding capture molecules,
sequencing of the enriched target regions of the two or more individuals, comprising acquisition of the marking,
assignment of the marking and therefore of the sequencing results to the individuals.

The selection of the target regions in the nucleic acid populations to be analyzed is effected with the aid of the medical diagnostic parameters to be determined. If information for cancer-relevant DNA or RNA regions is to be acquired by the method according to the invention, corresponding cancer-associated sequence regions (e.g. genes, exons, introns, transcripts) are selected. The selection of the corresponding sequence regions can be made with the aid of information known to the person skilled in the art or on the basis of corresponding data in databases, internet databases or genome projects. When the sequence regions have been selected, specific capture probes are provided for these regions. These capture probes have the task of picking out the predetermined regions from one or more/many complex nucleic acid populations. The selection of the capture probe preferably takes place with software assistance with the aid of further information available to persons skilled in the art or databases or internet databases. Such further information relates to e.g. complexity of the sequence (high- or low-complexity regions), length and fusion point of the capture probes, secondary structures of the capture probes or of the target regions, bonding affinities, specificities etc.

Other disease-associated regions (e.g. Alzheimer's disease, obesity, hypertension etc.) in the human genome can furthermore also be analyzed by the method according to the invention. The person skilled in the art recognizes, however, that the uses are not limited only to the human genome, but can also be employed on other organisms, e.g. mammals or other eukaryotic organisms or also prokaryotic or viral organisms.

A further a method for acquisition of information in the DNA or RNA of a number of at least two individuals comprises the steps:
selection of target regions in a DNA or RNA population,
preparation of nucleic acid populations of the individuals for a sequence enrichment with addition of a marking which later allows assignment to the individual,
preparation of a preparative biochip with a microarray of capture oligonucleotides, the sequence of which is selected to match the target regions,
sequence-specific enrichment of target regions from the nucleic acid populations of the two or more individuals in/on the preparative biochip, e.g. a microfluid biochip, with the capture molecules,
sequencing of the enriched target regions of the two or more individuals, comprising acquisition of the marking,
assignment of the marking and therefore of the sequencing results to the individuals.

A further method for acquisition of information in the DNA or RNA of a number of at least two individuals comprises the steps:
selection of target regions in a DNA or RNA population,
preparation of nucleic acid populations of the individuals for a sequence enrichment with addition of a marking which later allows assignment to the individual,
preparation of a preparative capture probe matrix, e.g. on beads or in the liquid phase, the sequence of which is selected to match the target regions,
sequence-specific enrichment of target regions from the nucleic acid populations of the two or more individuals on the preparative capture probe matrix, e.g. on beads or in the liquid phase,
sequencing of the enriched target regions of the two or more individuals, comprising acquisition of the marking,
assignment of the marking and therefore of the sequencing results to the individuals.

A further method for acquisition of information in the DNA or RNA of a number of at least two individuals comprises the steps:
selection of target regions in a DNA or RNA population,
preparation of nucleic acid populations of the individuals for a sequence enrichment with addition of a marking which later allows assignment to the individual,
preparation of a preparative biochip with a microarray of capture oligonucleotides, the sequence of which is filed in a database,
sequence-specific enrichment of target regions from the nucleic acid populations of the two or more individuals in/on the preparative biochip, e.g. a microfluid biochip, with corresponding capture molecules,
sequencing of the enriched target regions of the two or more individuals, comprising acquisition of the marking,
assignment of the marking and therefore of the sequencing results to the individuals.

A further a method for acquisition of information in the DNA or RNA of a number of at least two individuals comprises the steps:
selection of target regions in a DNA or RNA population,
preparation of nucleic acid populations of the individuals for a sequence enrichment with addition of a marking which later allows assignment to the individual,
preparation of a preparative capture probe matrix, e.g. on beads or in the liquid phase, the sequence of which is filed in a database,
sequence-specific enrichment of target regions from the nucleic acid populations of the two or more individuals on the preparative capture probe matrix, e.g. on beads or in the liquid phase, with corresponding capture molecules,
sequencing of the enriched target regions of the two or more individuals, comprising acquisition of the marking,
assignment of the marking and therefore of the sequencing results to the individuals.

The method according to the invention comprises processing (enrichment) of marked samples from individuals. This processing can be carried out by subjecting several or all of the samples to a parallel enrichment step. The method can furthermore provide for part amounts of the samples being processed in the “batch method”. The enriched samples can accordingly subsequently be subjected to sequence analysis of the enriched samples together or separately according to part amounts. Depending on the complexity of the sample and the nucleic acid regions to be enriched, it may be necessary to use one or more reaction chambers of the sequencing apparatus. That is to say the selection of the reaction chambers of the sequencing apparatus will be selected according to the complexity of the parameters or nucleic acid regions to be determined. Depending on the sequencing technology used, the sizes of the reaction chamber can be accordingly scaled down (454 and Solid by using frames/mats a larger reaction chamber is separated into small reaction chambers) and up (e.g. Roche-454, ABI-Solid, Illumina Genome Analyzer).

A method for acquisition of information in the DNA or RNA of a number of four or more individuals comprises the steps:
preparation of nucleic acid populations of the individuals for a sequence enrichment with addition of a marking which later allows assignment to the individual,
enrichment of the sample of each individual, e.g. in/on a preparative biochip (or on beads or in the liquid phase), with corresponding capture molecules,
sequencing of the enriched sample of two or more individuals, comprising acquisition of the marking, in one or more reaction chambers of a sequencing apparatus,
preparation of the sample of a further two or more individuals for a sequence enrichment with addition of a marking which later allows assignment to the individual,
enrichment of the sample of each individual, e.g. in/on a preparative biochip (or on beads or in the liquid phase),
sequencing of the enriched sample of two or more individuals comprising acquisition of the markings, in one or more reaction chambers of a sequencing apparatus,
assignment of the sequencing results to the individuals.

A method for acquisition of information in the DNA or RNA of a number of two and or more individuals comprises the steps:
preparation of nucleic acid populations of the individuals for a sequence enrichment with addition of a marking which later allows assignment to the individual,
enrichment of the sample of all the individuals, e.g. in/on a preparative biochip (or on beads or in the liquid phase), with corresponding capture molecules,
sequencing of the enriched sample of the two or more individuals, comprising acquisition of the marking in one or more reaction chambers of a sequencing apparatus,
assignment of the sequencing results to the individuals.

A method for acquisition of information in the DNA or RNA of a number of four or more individuals comprises the steps:
preparation of nucleic acid populations of the individuals for a sequence enrichment with addition of a marking which later allows assignment to the individual,
enrichment of the sample of a first part amount of the individuals, e.g. in/on a preparative biochip (or on beads or in the liquid phase),
consecutive enrichment of the sample in a second part amount of the individuals, e.g. on the same preparative biochip (or on the same beads or in the liquid phase),
sequencing of the enriched sample of the four or more individuals, comprising acquisition of the marking, in one or more reaction chambers of a sequencing apparatus,
assignment of the sequencing results to the individuals.

In a preferred embodiment, the capture probe matrix can be used several times. That is to say the capture probes can be purified or regenerated, so that one or more further enrichment cycles can be carried out on one and the same capture probe matrix. In a preferred embodiment, a preparative biochip is used as the capture matrix. Further embodiments of the capture probe matrix are capture probes immobilized on particles or beads or capture probe libraries in solution.

The number of enrichment cycles which can be carried out on one capture probe matrix is in principle not limited and is determined in the specific case by the number of possible diverse markings ((bar)codes available). If e.g. 16 (bar)codes are available, up to 16 analyses can be carried out consecutively on one and the same capture probe matrix. In the case of 100 (bar)codes, accordingly 100, and in the case of 1,000 (bar)codes then up to 1,000 analyses can be carried out.

Multiple marking of individual nucleic acids to be analyzed represents an extension of the diverse markings. Thus, the nucleic acids to be analyzed can have not only one marking, e.g. a terminal marking, but several terminal and additionally also one or more internal markings.

Since according to the invention the nucleic acid regions (DNA, RNA) of individuals which are to be enriched are provided with an individual-specific marking, in the event of multiple use of the capture probe matrix the data which originate from which individual can be clearly reconstructed. This is of quite decisive importance from quality aspects, since it must be ensured that above all the sequence data generated in a diagnostic context can be unambiguously assigned to an individual, and that residues of a preceding enrichment experiment can be ruled out from influencing the subsequent analysis or from being falsely added to the data set of the subsequent analysis. The present method is therefore an innovatively integrated mode of approach both from the point of view of cost and with respect to the requirement of quality assurance/quality of the data.

A further method for acquisition of information in the DNA or RNA of a number of four or more individuals comprises the steps:
preparation of nucleic acid populations of the individuals for a sequence enrichment with addition of a marking which later allows assignment to the individual,
enrichment of the sample of a first part amount of the individuals, e.g. in/on a preparative biochip (or on beads or in the liquid phase), with corresponding capture molecules,
purification of the preparative biochip (the beads or the capture probes for the enrichment in the liquid phase),
consecutive enrichment of the sample in a second part amount of the individuals in/on the same preparative biochip (or on the same beads or in the liquid phase),
sequencing of the enriched sample of the four or more individuals, comprising acquisition of the marking, in one or more reaction chambers of a sequencing apparatus,
assignment of the sequencing results to the individuals.

A further method for acquisition of information in the DNA or RNA of a number of four or more individuals comprises the steps:
preparation of nucleic acid populations of the individuals for a sequence enrichment with addition of a marking which later allows assignment to the individual,
enrichment of the sample of a first part amount of the individuals, e.g. in/on a preparative biochip (or on beads or in the liquid phase), with corresponding capture molecules,
regeneration of the preparative biochip (the beads or the capture probes for enrichment in the liquid phase),
consecutive enrichment of the sample of a second part amount of the individuals, e.g. in/on the same preparative biochip (or on the same beads or in the liquid phase),
sequencing of the enriched sample of the four or more individuals, comprising acquisition of the marking, in one or more reaction chambers of a sequencing apparatus,
assignment of the sequencing results to the individuals.

A further method for acquisition of information in the DNA or RNA of a number of four or more individuals comprises the steps:
preparation of nucleic acid populations of the individuals for a sequence enrichment with addition of a marking which later allows assignment to the individual,
enrichment of the sample of a first part amount of the individuals, e.g. in/on a preparative biochip (or on beads or in the liquid phase), with corresponding capture molecules,
consecutive enrichment of the sample of a second part amount of the individuals, e.g. in/on the same preparative biochip (or on the same beads or the same capture probes for the enrichment in the liquid phase)
sequencing of the enriched sample of the four or more individuals, comprising acquisition of the marking in one or more reaction chambers of a sequencing apparatus
assignment of the sequencing results to the individuals,
determination of the rate of entrainment of nucleic acids from the first and the consecutive enrichment step using the sequencing results and the markings.

A further method for acquisition of information in the DNA or RNA of a number of four or more individuals comprises the steps:
preparation of nucleic acid populations of the individuals for a sequence enrichment with addition of a marking which later allows assignment to the individual,
enrichment of the sample of a first part amount of the individuals, e.g. in/on a preparative biochip (or on beads or in the liquid phase), with corresponding capture molecules,
sequencing of the enriched sample of the first part amount of the individuals, comprising acquisition of the marking,
consecutive enrichment of the sample of a second part amount of the individuals, e.g. in/on the same preparative biochip (or on the same beads or the same capture probes for the enrichment in the liquid phase),
sequencing of the enriched sample of the four or more individuals, comprising acquisition of the marking,
assignment of the sequencing results to the individuals,
determination of the rate of entrainment of nucleic acids from the first and the consecutive enrichment step using the sequencing results and the markings.

A further method for acquisition of information in the DNA or RNA of a number of four or more individuals in two or more laboratories comprises the steps:
preparation of nucleic acid populations of the individuals for a sequence enrichment with addition of a marking which later allows assignment to the individual and to the laboratory,
sequencing of the samples of the individuals, comprising acquisition of the marking,
assignment of the sequencing results to the individuals and the laboratories.

A further method for acquisition of information in the DNA or RNA of a number of four or more individuals in two or more laboratories comprises the steps:
preparation of nucleic acid populations of the individuals for a sequence enrichment with addition of a marking which later allows assignment to the individual and to the laboratory,
sequencing of the samples of the individuals, comprising acquisition of the marking,
assignment of the sequencing results to the individuals and the laboratories,
storage of the sequencing results and/or the markings for the purpose of quality control and/or quality assurance.

A further method for acquisition of information in the DNA or RNA of a number of four or more individuals in two or more laboratories comprises the steps:
preparation of nucleic acid populations of the individuals for a sequence enrichment with addition of a marking which later allows assignment to the individual and to the laboratory,
sequencing of the samples of the individuals, comprising acquisition of the marking,
assignment of the sequencing results to the individuals and laboratories,
deriving of individual diagnostic information from the sequencing results,
storage of the markings for the purpose of quality control and/or quality assurance.

A further method for acquisition of information in the DNA or RNA of a number of four or more individuals in two or more laboratories comprises the steps:
preparation of nucleic acid populations of the individuals for a sequence enrichment with addition of a marking which later allows assignment to the individual and to the laboratory,
sequencing of the samples of the individuals, comprising acquisition of the marking,
assignment of the sequencing results to the individuals and laboratories,
deriving of individual diagnostic information and/or individual recommendations from the sequencing results.

A further method for acquisition of information in the DNA or RNA of a number of four or more individuals in two or more laboratories comprises the steps:
preparation of nucleic acid populations of the individuals for a sequence enrichment with addition of a marking which later allows assignment to the individual and to the laboratory,
sequencing of the samples of the individuals, comprising acquisition of the marking,
assignment of the sequencing results to the individuals and laboratories,
deriving of recommendations for action for the therapy of one or more of the individuals.

A further method for acquisition of information in the DNA or RNA of a number of two or more individuals on two or more sequencing apparatuses comprises the steps:
preparation of nucleic acid populations of the individuals for a sequence enrichment with addition of a marking which later allows assignment to the individual and to the sequencing apparatus,
sequencing of the samples of the individuals, comprising acquisition of the marking,
assignment of the sequencing results to the individuals and to the sequencing apparatuses.

A further method for acquisition of information in the DNA or RNA of a number of six or more individuals on two or more sequencing apparatuses in two or more laboratories comprises the steps:
preparation of nucleic acid populations of the individuals for a sequence enrichment with addition of a marking which later allows assignment to the individual, to the sequencing apparatus and to the laboratory,
sequencing of the samples of the individuals, comprising acquisition of the marking,
assignment of the sequencing results to the individuals, to the sequencing apparatuses and to the laboratories,
storage of the markings and/or the sequencing results and/or the assignments, e.g. for the purpose of quality control and/or quality assurance.

A further method for acquisition of information in the DNA or RNA of a number of four or more individuals in two or more laboratories comprises the steps:
preparation of nucleic acid populations of the individuals for a sequence enrichment with addition of a marking which later allows assignment to the individual and to the laboratory,
enrichment of the nucleic acid populations of the individuals, e.g. in/on a preparative biochip (or on beads or in liquid phase) using suitable capture molecules,
sequencing of the samples of the individuals, comprising acquisition of the marking,
assignment of the sequencing results to the individuals and laboratories.

A further method for acquisition of information in the DNA or RNA of a number of four or more individuals in two or more laboratories comprises the steps:
preparation of nucleic acid populations of the individuals for a sequence enrichment with addition of a marking which later allows assignment to the individual and to the laboratory,
enrichment of the nucleic acid populations of the individuals, e.g. in/on a preparative biochip (or on beads or liquid phase) using suitable capture molecules,
sequencing of the samples of the individuals, comprising acquisition of the marking,
assignment of the sequencing results to the individuals and laboratories,
storage of the sequencing results and/or the markings for the purpose of quality control and/or quality assurance.

A further method for acquisition of information in the DNA or RNA of a number of four or more individuals in two or more laboratories comprises the steps:
preparation of nucleic acid populations of the individuals for a sequence enrichment with addition of a marking which later allows assignment to the individual and to the laboratory,
enrichment of the nucleic acid populations of the individuals, e.g. in/on a preparative biochip (or on beads or liquid phase) using suitable capture molecules,
sequencing of the samples of the individuals, comprising acquisition of the marking, assignment of the sequencing results to the individuals and laboratories,
deriving of individual diagnostic information from the sequencing results,
storage of the markings for the purpose of quality control and/or quality assurance.

A further method for acquisition of information in the DNA or RNA of a number of four or more individuals in two or more laboratories comprises the steps:
preparation of nucleic acid populations of the individuals for a sequence enrichment with addition of a marking which later allows assignment to the individual and to the laboratory,
enrichment of the nucleic acid populations of the individuals, e.g. in/on a preparative biochip (or on beads or liquid phase) using suitable capture molecules,
sequencing of the samples of the individuals, comprising acquisition of the marking,
assignment of the sequencing results to the individuals and laboratories,
deriving individual diagnostic information and/or individual recommendations from the sequencing results.

A further method for acquisition of information in the DNA or RNA of a number of four or more individuals in two or more laboratories comprises the steps:
preparation of nucleic acid populations of the individuals for a sequence enrichment with addition of a marking which later allows assignment to the individual and to the laboratory,
enrichment of the nucleic acid populations of the individuals, e.g. in/on a preparative biochip (or on beads or liquid phase) using suitable capture molecules,
sequencing of the samples of the individuals, comprising acquisition of the marking,
assignment of the sequencing results to the individuals and laboratories,
deriving recommendations for action for the therapy of one or more of the individuals.

A further method for acquisition of information in the DNA or RNA of a number of two or more individuals on two or more sequencing apparatuses comprises the steps:
preparation of nucleic acid populations of the individuals for a sequence enrichment with addition of a marking which later allows assignment to the individual and to the sequencing apparatus,
enrichment of the nucleic acid populations of the individuals, e.g. in/on a preparative biochip (or on beads or liquid phase) using suitable capture molecules,
sequencing of the samples of the individuals, comprising acquisition of the marking,
assignment of the sequencing results to the individuals and to the sequencing apparatuses.

A further method for acquisition of information in the DNA or RNA of a number of six or more individuals on two or more sequencing apparatuses in two or more laboratories comprises the steps:
preparation of nucleic acid populations of the individuals for a sequence enrichment with addition of a marking which later allows assignment to the individual, to the sequencing apparatus and to the laboratory,
enrichment of the nucleic acid populations of the individuals, e.g. in/on a preparative biochip (or on beads or liquid phase) using suitable capture molecules,
sequencing of the samples of the individuals, comprising acquisition of the marking,
assignment of the sequencing results to the individuals, to the sequencing apparatuses and to the laboratories,
storage of the markings and/or the sequencing results and/or the assignments, e.g. for the purpose of quality control and/or quality assurance.

In a preferred embodiment, the steps of enrichment and sequence analysis are combined and carried out in an integrated installation. This has the advantage that the corresponding analyses can be carried out in a highly automated and integrated manner. The system limits and therefore harmful influences of operating or handling errors are reduced by this means. This has a direct influence on the error rates of the measurements and therefore has a positive effect on the quality of the corresponding analyses. This is of decisive importance above all in the field of diagnostics, e.g. clinical diagnostics.

The invention therefore also relates to an installation for acquisition of information in the DNA or RNA of an individual by sequence-specific enrichment of target regions of the DNA or RNA in/on a capture probe matrix, e.g. a preparative biochip, comprising
a capture probe matrix,
a device for loading the capture probe matrix with a DNA or RNA sample,
a device for feeding reagents for washing the capture probe matrix,
a device for elution of an enriched DNA or RNA sample from the capture probe matrix,
one or more sequencing reaction chambers,
a device for loading the one or more sequencing reaction chambers
a device for carrying out a parallel sequencing reaction in the sequencing reaction chambers, e.g. by means of sequencing-by-synthesis or by means of sequencing-by-ligation,
a memory-programmable device for carrying out the parallel sequencing reaction,
a memory-programmable device and a storage medium for storage of the sequencing results,
optionally a device for the amplification of the DNA or RNA sample (before the enrichment step and/or after the enrichment step).

According to the invention, multiplication or amplification of the sample to be analyzed or the enriched sample may be necessary. This is important above all in the cases where either insufficient starting material is available for the enrichment, or insufficient material to carry out the subsequent sequence analysis is obtained after the enrichment. The amplification of the starting material or the amplification of the enriched material can be integrated here into the processing of the capture probe matrix, e.g. of a preparative biochip, beads or capture probes in solution, and therefore into the enrichment installation. The amplification of the enriched material can also be integrated into the processing of the sequence analysis and therefore into the sequencing installation.

The amplification may be carried out either isothermally or by thermocycling. The device for amplification may comprise a reaction temperature control unit which may be regulated by thermoelements, Peltier elements or by other principles/technologies known to the skilled person (from the field of the construction of PCR and RT-PCR devices).

The amplification may be used for the multiplication of the starting sample (DNA or RNA sample, respectively) and/or for the multiplication of the enriched sample before it is subjected to sequence analysis).

If an enrichment is carried out over several cycles of enrichment, a multiplication of the eluted enriched material may be effected in each case before the subsequent cycle in order to provide sufficient starting material in the subsequent enrichment cycle. In a further preferred embodiment, the multiplication or amplification of the sample to be analyzed or the enriched sample takes place in an integrated manner in the integrated installation described for the for enrichment and sequencing. This is important above all in the cases where either insufficient starting material is available for the enrichment, or insufficient material to carry out the subsequent sequence analysis is obtained after the enrichment.

The invention therefore also relates to an installation for acquisition of information in the DNA or RNA of an individual by sequence-specific enrichment of target regions of the DNA or RNA in/on a capture probe matrix, e.g. a preparative biochip, comprising
a capture probe matrix,
a device for loading the capture probe matrix with a DNA or RNA sample,
a device for feeding reagents for washing the capture probe matrix,
a device for elution of the enriched DNA or RNA sample from the capture probe matrix,
one or more sequencing supports,
a device for loading the one or more sequencing supports in the form of beads, microbeads or microparticles,
a device for loading a support or a flow cell with the beads, microbeads or microparticles,
a device for carrying out a parallel sequencing reaction, e.g. by means of sequencing-by-synthesis or by means of sequencing-by-ligation,
a memory-programmable device for carrying out the parallel sequencing reaction,
a memory-programmable device and a storage medium for storage of the sequencing results.

### EXAMPLES

### Example 1

### Multiplexing of Genome Analyses

| Markings | | | | |
|---|---|---|---|---|
| (bar | Enrichment | # | Illumina | # |
| codes), | s-matrix, | individuals/ | NGS, | individuals/ |
| plex | plex | day | plex | 3 days |
| 8 | 8 | 64 | 8 | 64 |
| 24 | 8 | 192 | 8 | 192 |
| 48 | 8 | 384 | 8 | 384 |
| 96 | 8 | 768 | 8 | 768 |
| 8 | 32 | 256 | 32 | 256 |
| 24 | 32 | 768 | 32 | 768 |
| 48 | 32 | 1536 | 32 | 1536 |
| 96 | 32 | 3072 | 32 | 3072 |

If 24 markings (bar codes) are used, target regions can be isolated from the genome for 192 individuals in total if an enrichment matrix which renders possible 8 independent enrichment experiments per day in parallel is used. These are subsequently analyzed within 3 days on an Illumina next generation sequencing apparatus which allows eight analyses in parallel. That is to say the medical parameters of 192/3=64 individuals can be determined per day through the pipeline. If 3 Illumina NGS are used instead, 192 individuals can be analyzed per day.

### Example 2

### Incorporation of a Barcode into a Sequencing Library Implementing Restriction Enzyme Xcml

The recognition sequence and the cleavage site (arrow) of XcmI are as follows:

| | |
|---|---|
| | ↓ |
| Xcml: | CCANNNNN NNNNTGG |

Cleavage with XcmI generates a single nucleotide (N)-3′-overhang.

The standard library preparation procedure for the Illumina sequencing platform includes fragmenting the genomic DNA, end-repair and adding a 3′-A-overhang.

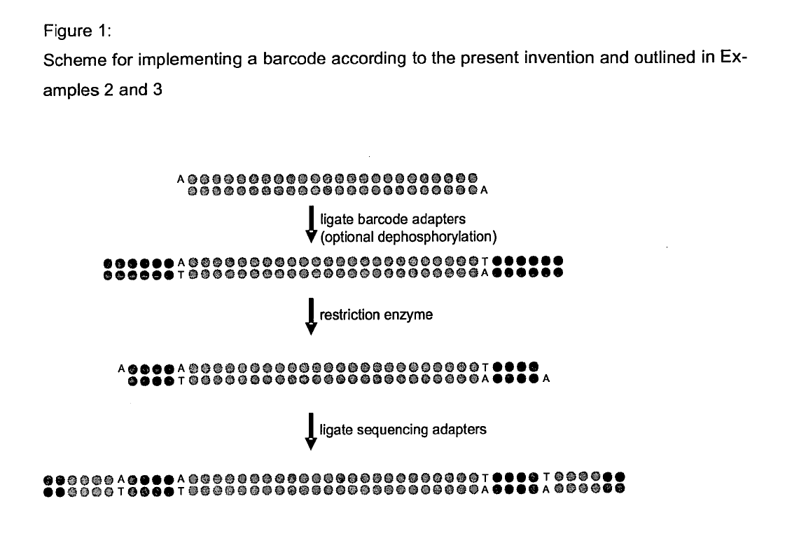

In order to comply with this a procedure for implementing a barcode adaptor comprising the following Steps 1-4 was performed. This procedure is schematically depicted in FIG. 1.

Step 1: Providing a barcode adaptor nucleic acid with the following sequence:

5′ X_{y}CCANNNNTnnnnTGGn_{z}T 3′
3′ X_{y}GGTNNNNAnnnnACCn_{z}P 5′

wherein
N=in each case independently any possible nucleotide (A, C, G, T, I, . . . ) on the first strand and a complementary nucleotide on the opposite strand
n=in each case independently any possible nucleotide (A, C, G, T, I, . . . ) on the first strand and a complementary nucleotide on the opposite strand
z=an integer (0, 1, 2, 3, e.g. up to 30))
P=a phosphorylation or phosphate group
X=in each case independently any possible nucleotide (A, C, G, T, I, . . . ) on the first strand, and a complementary nucleotide on the opposite strand
y=an integer (0, 1, 2, 3, e.g. up to 50).

Hereby represents “n” the barcode positions. For z=0 the barcode adaptor includes 4 base positions, resulting in 4 to power of 4 possible barcodes=256 barcodes. If z=2, a number of up to 4096 barcodes is possible.

The adaptor oligonucleotides can be prepared synthetically. They have preferably a length of 18-45 nucleotides.

Step 2: Ligation of the barcode adaptor to the fragmented library:

The fragmented sequencing library contains a 3′-A-overhang that was created after fragmentation, and end repair when producing the sequencing library according to the standard procedure.

| | |
|---|---|
| X_{y}CCANNNNTnnnnTGGn_{z}T | NNNNNNN (sequencing library) |
| X_{y}GGTNNNNAnnnnACCn_{z}P | ANNNNNNN(sequencing library) |

Due to the 3′-A-overhang on the sequencing library and the 3′-T-overhang on the barcode adaptor, a directed ligation (TA-cloning) ensures a high yield.

X_{y}CCANNNNTnnnnTGG [symbol] TNNNNNNN(sequencing library)
X_{y}GGTNNNNAnnnnACC [symbol] ANNNNNNN(sequencing library)

Optionally a dephosphorylation step is incorporated after the ligation step. This step removes phosphorylation from fragments of the sequencing library and prevents that these molecules—which do not contain a barcode adaptor—are subject to ligation to the sequencing adaptor in step 4.

Step 3: Restriction digestion with Xcml The ligated construct of Step 2 is treated with Xcml to produce:

nnnnTGG [symbol] TNNNNNNN(sequencing library)
AnnnnACC [symbol] ANNNNNNN(sequencing library)

Step 4: Ligation of the sequencing adaptor

5′ (adaptor)-
NNNT nnnnTGG [symbol] TNNNNNNN(sequencing library)
3′ (adaptor)-
NNN AnnnnACC [symbol] ANNNNNNN(sequencing library)

The standard sequencing adaptor has a T-overhang at the 3′-end. Ligation to the construct of Step 3 having an A-overhang results in high yields:

5′ (adaptor)-
NNNTnnnnTGG [symbol] TNNNNNNN-(sequencing library) 3′
3′ (adaptor)-
NNNAnnnnACC [symbol] ANNNNNNN-(sequencing library) 5′

For simplicity, only one end of the DNA library fragment is shown. Following the outlined scheme, barcode adaptors and sequencing adaptors may be ligated to both ends of the sequence library fragments.

Till now, barcodes on the Illumina sequencing platform have to be read by a second sequencing run with a separate primer, making it much more cumbersome, error-prone and expensive compared to a simple single read-run enabled by the present invention.

The strategy of the present invention allows for a 75 bp or 100 bp single-read sequencing run with up to 256 barcodes at the terminal end of the library fragments combined with a fixed TnnnnTGGn_{z}T-sequence motif (and its complement) which can be nicely employed as a QC-criterium for filtering during sequence data analysis. This leaves 67 to 92 bp of the fragment of 75 bp or 100 bp sequence reads for mapping.

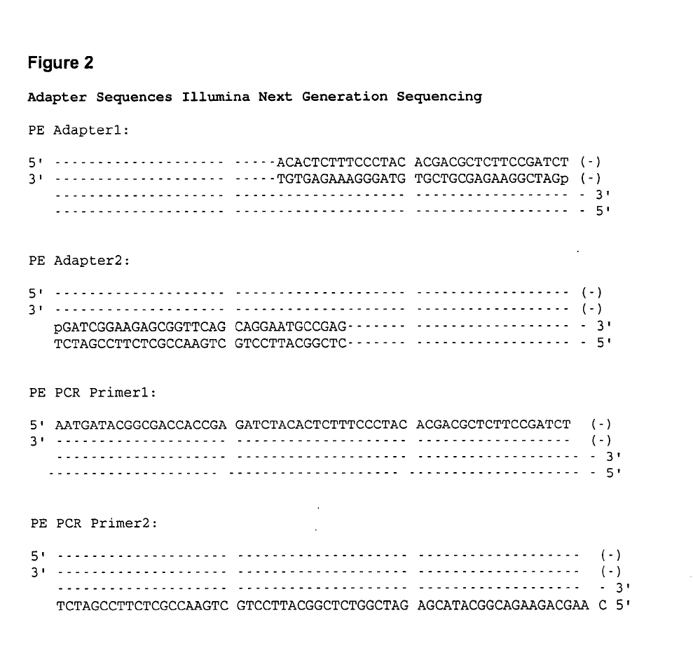

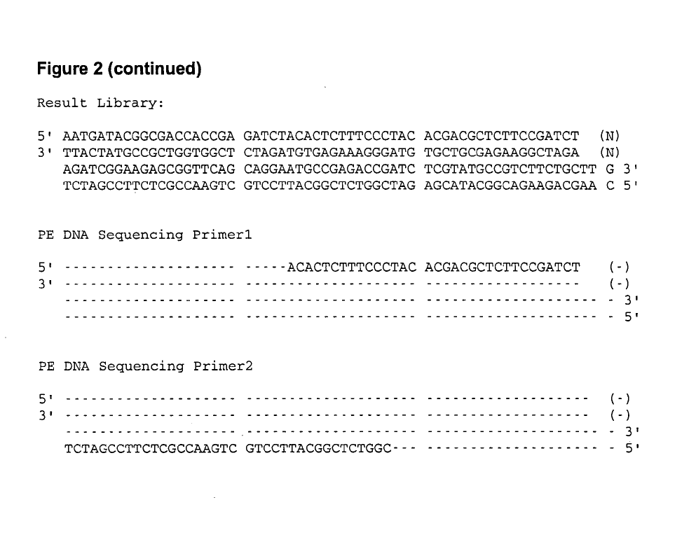

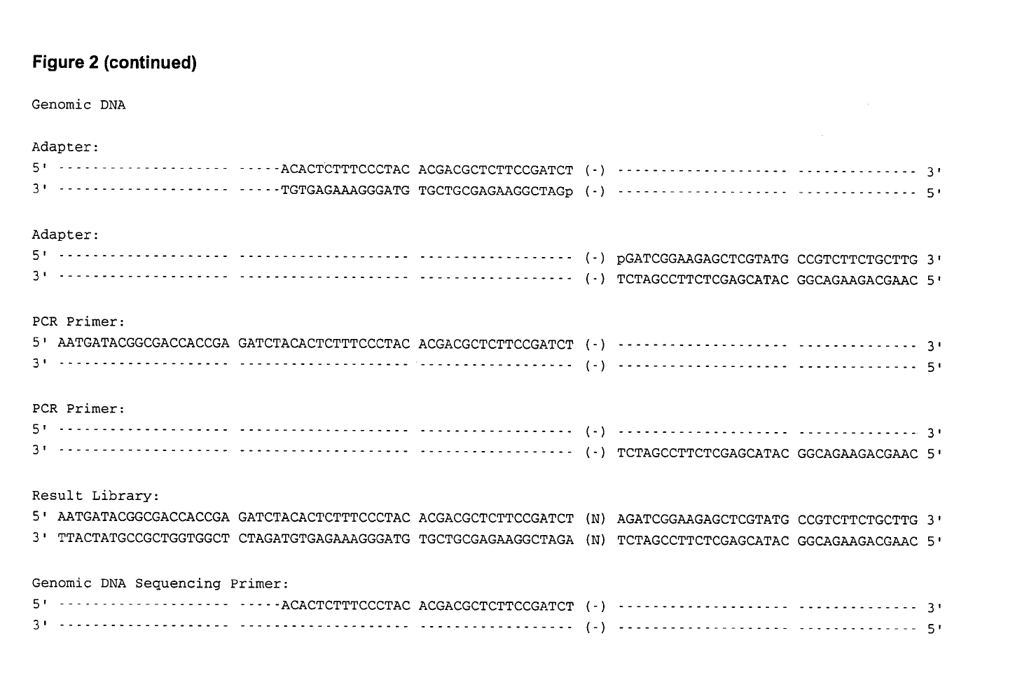

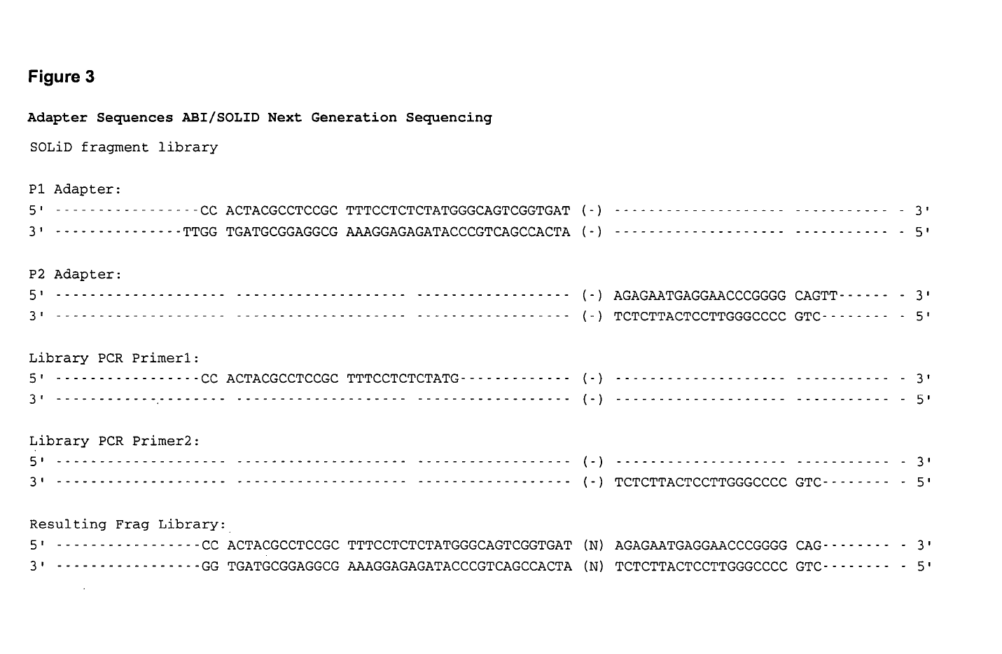

Although this procedure is described for the Illumina sequencing platform, the person skilled in the art will recognize that this way of implementing barcodes into a sequencing library is also applicable to any other sequencing platform (e.g. ABI Solid, Roche 454, etc.). The person skilled in the art will be able to select the appropriate sequencing adaptor sequences for the relevant sequencing platform. Suitable adaptor sequences are shown in FIG. 2 for the Illumina platform and in FIG. 3 for the ABI/SOLID platform.

In a preferred embodiment related to Example 2, the barcode adaptor sequences include additional nucleotides Zₖ wherein k is preferably up to 20, e.g. 1, 2, 3 or 4, at the 5′ end in order to prevent the formation of undesired products during ligation.

Thus, preferred barcode adaptors of the invention have the following sequence:

5′ZₖX_{y}CCANNNNTnnnnTGG [symbol] T 3′
3′ X_{y}GGTNNNNAnnnnACC [symbol] P 5′

wherein
N=in each case independently any possible nucleotide (A, C, G, T, I, . . . ) on the first strand and a complementary nucleotide on the opposite strand
n=in each case independently any possible nucleotide (A, C, G, T, I, . . . ) on the first strand and a complementary nucleotide on the opposite strand
z=an integer: (0, 1, 2, 3, e.g. up to 30)
P=a phosphorylation or phosphate group
X=in each case independently any possible nucleotide (A, C, G, T, I, . . . ) on the first strand and a complementary nucleotide on the opposite strand
y=an integer (0, 1, 2, 3, e.g. up to 50)
Z=in each case independently any possible nucleotide (A, C, G, T, I, . . . )_{—}
k=an integer (0, 1, 2, 3, e.g. up to 20)

Preferably k=1 and Z=T or C or G, more preferably k=2 and Z=T or C or G or A, and most preferably k=2 and Z=T.

### Example 3

### Incoporation of a Barcode into a Sequencing Library Implementing Restriction Enzyme Eam1105I

The recognition sequence of Eam1105I (or its isoschizomers AhdI, AspEI, BmeRI, DriI, and EclHKI) is as follows:

↓
GACNNN NNGTC

Cleavage with Eam1105I or its isoschizomers generates a single nucleotide (N) 3′-overhang.

The standard library preparation procedure for the Illumina sequencing platform includes fragmenting the genomic DNA, end-repair and adding a 3′-A.

In order to comply with this, a procedure for implementing a barcode adaptor comprising the following Steps 1-4 was performed. This procedure is schematically depicted in FIG. 1.

Step 1: Providing a barcode adaptor with the following sequence:

5′-X_{y}GACNNTnnGTC [symbol] T - 3′
3′-X_{y}CTGNNAnnCAG [symbol] P - 5′

wherein
N=in each case independently any possible nucleotide (A, C, G, T, I, . . . ) on the first strand and a complementary nucleotide on the opposite strand
n=in each case independently any possible nucleotide (A, C, G, T, I, . . . ) on the first strand and a complementary nucleotide on the opposite strand
z=an integer (0, 1, 2, 3, e.g. up to 30)
P=a phosphorylation or phosphate group,
X=in each case independently any possible nucleotide (A, C, G, T, I, . . . ) on the first strand and a complementary nucleotide on the opposite strand
y=an integer (0, 1, 2, 3, e.g. up to 50)

Hereby represents “n” the barcode positions. For z=0 the barcode adaptor includes 2 base positions, resulting in 4 to power of 2 possible barcodes=16 barcodes. If z=2, a number of up to 256 barcodes is possible.

The adaptor oligonucleotides can be prepared synthetically. They have preferably a length of 12-45 nucleotides.

Step 2: Ligation of the barcode adaptor to the fragmented library:

The fragmented sequencing library contains a 3′-A-overhang that was created after fragmentation, and end repair when producing the sequencing library according to the standard procedure.

5′-X_{y}GACNNTnnGTC [symbol] T NNNNNNN(sequencing library)
3′-X_{y}CTGNNAnnCAG [symbol] P ANNNNNNN(sequencing library)

Due to the 3′-A-overhang on the sequencing library and the 3′-T-overhang on the barcode adaptor, a directed ligation (TA-cloning) ensures a high yield:

5′-X_{y}GACNNTnnGTC [symbol] TNNNNNNN(sequencing library)
3′-X_{y}CTGNNAnnCAG [symbol] ANNNNNNN(sequencing library)

Optionally a dephosphorylation step is incorporated after the ligation step. This step removes phosphorylation from fragments of the sequencing library and prevents that these molecules—which do not contain a barcode adaptor—are subject to ligation to the sequencing adaptor in step 4.

Step 3: Restriction digestion with Eam1105I

The ligated construct of Step 2 is treated with Eam1105I to produce:

5′- nnGTC [symbol] TNNNNNNN(sequencing library)
3′-AnnCAG [symbol] ANNNNNNN(sequencing library)

Step 4: Ligation of the sequencing adaptor

5′(adaptor)-
NNNT nnGTC [symbol] TNNNNNNN(sequencing library)
3′(adaptor)-
NNN AnnCAG [symbol] ANNNNNNN(sequencing library)

The standard sequencing adaptor has a T-overhang at the 3′-end. Ligation to the construct of Step 3 having an 3′-A-overhang results in high yields:

5′(adaptor)-
NNNTnnGTC [symbol] TNNNNNNN(sequencing library)
3′(adaptor)-
NNNAnnCAG [symbol] ANNNNNNN(sequencing library)

For simplicity, only one end of the DNA library fragment is shown. Following the outlined scheme, barcode adaptors and sequencing adaptors may be ligated to both ends of the sequence library fragments.

Till now, barcodes on the Illumina sequencing platform have to be read by a second sequencing run with a separate primer, making it much more cumbersome, error-prone and expensive compared to a single read-run enabled by the present invention.

The strategy of the present invention allows for a 75 bp or 100 bp single-read sequencing run with up to 256 barcodes at the terminal end of the library fragments combined with a fixed TnnGTCn_{z}T-sequence motif (and its complement) which can be nicely employed as a QC-criterium for filtering during sequence data analysis. This leaves 67 to 92 bp of the fragment of 75 bp or 100 bp sequence reads for mapping.

Although this procedure is described for the Illumina sequencing platform, the person skilled in the art will recognize that this way of implementing barcodes into a sequencing library is also applicable to any other sequencing platform (e.g. ABI Solid, Roche 454, etc.). The person skilled in the art will be able to select the appropriate sequencing adaptor sequences for the relevant sequencing platform. Suitable adaptor sequences are shown in FIG. 2 for the Illumina platform and in FIG. 3 for the ABI/SOLID platform.

Due to the fact that the barcode adaptors can be symetrically added to both sides of the fragment library molecules one embodiment of the invention envisions that only one or alternatively both adaptors are read out by the sequencing analysis. In case when both barcode adaptors are read out one can function to double-check the other.

In a special embodiment related to Example 3, the barcode adaptor sequences include additional nucleotides Zₖ wherein k is preferably an integer up to 20, e.g. 1, 2, 3 or 4, at the 5′-end in order to prevent the formation of undesired products during ligation

Thus, preferred barcode adaptors of the invention have the following sequence:

5′-ZₖX_{y}GACNNTnnGTC [symbol] T - 3′
3′- X_{y}CTGNNAnnCAG [symbol] P - 5′

wherein
N=in each case independently any possible nucleotide (A, C, G, T, I, on the first strand and a complementary nucleotide on the opposite strand
n=in each case independently any possible nucleotide (A, C, G, T, I, . . . ) on the first strand and a complementary nucleotide on the opposite strand
z=an integer (0, 1, 2, 3, e.g. up to 30)
P=a phosphorylationor phosphate group
X=in each case independently any possible nucleotide (A, C, G, T, I, . . . ) on the first strand and a complementary nucleotide on the opposite strand
y=an integer (0, 1, 2, 3, e.g. up to 50)
Z=in each case independently any possible nucleotide (A, C, G, T, I, . . . )
k=an integer (0, 1, 2, 3, e.g. up to 20)

Preferably k=2 and Z=T or C or G or A.

1. A method for isolation of target nucleic acid molecules, comprising the steps:
(a) providing one or more nucleic acid molecule populations to be analyzed,
(b) introducing markings into the nucleic acid populations to be analyzed,
(c) bringing the one or more populations of nucleic acid molecules into contact with capture molecules under conditions under which target nucleic acid molecules from the population or populations to be analyzed can bind specifically to the capture molecules,
(d) separating off material not bound to capture molecules and
(e) isolating and optionally characterizing the target nucleic acid molecules, comprising determination of the markings.

2. The method as claimed in claim 1, characterized in that a parallel determination of nucleic acid molecules which each carry a different marking is carried out.

3. The method as claimed in claim 1, characterized in that several populations of nucleic acid molecules which originate from different individuals of a species are analyzed.

4. The method as claimed in claim 1, characterized in that the capture molecules are immobilized on a support, e.g. on an array, a biochip or on particles.

5. The method as claimed in claim 1, characterized in that the capture molecules are present in the free form.

6. The method as claimed in claim 1, characterized in that the marking comprises a detectable group.

7. The method as claimed in claim 1, characterized in that the marking comprises one or more terminal adaptor sequences.

8. The method as claimed in claim 1, characterized in that an assignment to specific individuals, laboratories and/or sequencing apparatuses is made possible by the marking.

9. The method as claimed in claim 1, characterized in that it comprises several successive isolation cycles using the same or different capture molecules.

10. The method as claimed in claim 1, characterized in that after an isolation cycle has been carried out, the capture molecules are purified and re-used in one or more subsequent isolation cycles for target nucleic acid molecules.

11. The method as claimed in claim 10, characterized in that capture molecules immobilized on a support, in particular a biochip, are re-used.

12. The method as claimed in claim 1, characterized in that a marking comprises a sequence inserted between the target nucleic acid molecules and a sequencing adaptor.

13. The method as claimed in claim 12, characterized in that the marking comprises the following sequence:

| | |
|---|---|
| 5′ ZₖX_{y}CCANNNNTnnnnTGGn_{z}T 3′ | (SEQ ID NO.: 4) |
| 3′ X_{y}GGTNNNNAnnnnACCn_{z}P 5′ | (SEQ ID NO.: 5) |

wherein
N=in each case independently any possible nucleotide (A, C, G, T, I, . . . ) on the first strand and a complementary nucleotide on the opposite strand
n=in each case independently any possible nucleotide (A, C, G, T, I, . . . ) on the first strand and a complementary nucleotide on the opposite strand
z=an integer: (0, 1, 2, 3, e.g. up to 30)
P=a phosphorylation or phosphate group
X=in each case independently any possible nucleotide (A, C, G, T, I, . . . ) on the first strand and a complementary nucleotide on the opposite strand
y=an integer (0, 1, 2, 3, e.g. up to 50)
Z=in each case independently any possible nucleotide (A, C, G, T, I, . . . )
k=an integer (0, 1, 2, 3, e.g. up to 20).

14. The method as claimed in claim 12, characterized in that the marking comprises the following sequence:

| | |
|---|---|
| 5′-ZₖX_{y}GACNNTnnGTCn_{z}T - 3′ | (SEQ ID NO.: 9) |
| 3′- X_{y}CTGNNAnnCAGn_{z}P - 5′ | (SEQ ID NO.: 10) |

wherein
N=in each case independently any possible nucleotide (A, C, G, T, I, . . . ) on the first strand and a complementary nucleotide on the opposite strand
n=in each case independently any possible nucleotide (A, C, G, T, I, . . . ) on the first strand and a complementary nucleotide on the opposite strand
z=an integer (0, 1, 2, 3, e.g. up to 30)
P=a phosphorylationor phosphate group
X=in each case independently any possible nucleotide (A, C, G, T, I, . . . ) on the first strand and a complementary nucleotide on the opposite strand
y=an integer (0, 1, 2, 3, e.g. up to 50)
Z=in each case independently any possible nucleotide (A, C, G, T, I, . . . )
k=an integer (0, 1, 2, 3, e.g. up to 20).

15. An apparatus for acquisition of information in the DNA or RNA of an individual by sequence-specific enrichment of target regions of the DNA or RNA in/on a capture probe matrix, e.g. a preparative biochip, comprising
a capture probe matrix,
a device for loading the capture probe matrix with a DNA or RNA sample,
a device for feeding reagents for washing the capture probe matrix,
a device for elution of an enriched DNA or RNA sample from the capture probe matrix,
one or more sequencing reaction chambers,
a device for loading the one or more sequencing reaction chambers
a device for carrying out a parallel sequencing reaction in the sequencing reaction chambers, e.g. by means of sequencing-by-synthesis or by means of sequencing-by-ligation,
a memory-programmable device for carrying out the parallel sequencing reaction,
a memory-programmable device and a storage medium for storage of the sequencing results.

16. An apparatus for acquisition of information in the DNA or RNA of an individual by sequence-specific enrichment of target regions of the DNA or RNA in/on a preparative biochip, comprising
a capture probe matrix,
a device for loading the capture probe matrix with a DNA or RNA sample,
a device for feeding reagents for washing the capture probe matrix,
a device for elution of the enriched DNA or RNA sample from the capture probe matrix,
one or more sequencing supports,
a device for loading the one or more sequencing supports in the form of beads, microbeads or microparticles,
a device for loading a support or a flow cell with the beads, microbeads or microparticles,
a device for carrying out a parallel sequencing reaction, e.g. by means of sequencing-by-synthesis or by means of sequencing-by-ligation,
a memory-programmable device for carrying out the parallel sequencing reaction,
a memory-programmable device and a storage medium for storage of the sequencing results.
